(19) United States
(12) Patent Application Publication
(10) Pub. No.: US 2025/0248739 A1
(43) Pub. Date: Aug. 7, 2025

(54) FLUTED AND FENESTRATED INTRAMEDULLARY DEVICES

(71) Applicant: Reselute, Inc., Durham, NC (US)

(72) Inventors: Cambre KELLY, Durham, NC (US); Jake DONOVAN, Durham, NC (US); Fioleda Kesseli, Durham, NC (US); Jeremy Blair, Atlanta, GA (US)

(21) Appl. No.: 19/042,261

(22) Filed: Jan. 31, 2025

Related U.S. Application Data
(60) Provisional application No. 63/549,423, filed on Feb. 2, 2024.

(51) Int. Cl. A61B 17/34; A61B 17/56; A61B 17/72

(52) U.S. Cl. CPC A61B 17/3472; A61B 17/7283; A61B 2017/561

(57) ABSTRACT

Fluted and fenestrated implant devices are discussed herein. In various embodiments, the orthopedic implant device includes an external surface having a plurality of fenestrations, and one or more radially distributed concave longitudinal flutes oriented parallel to the longitudinal axis. The longitudinal flutes may include a bottom surface and a plurality of fenestrations through the bottom surface. The implant device may also include a central reservoir filled with a temporary carrier and connected to the plurality of fenestrations, wherein the orthopedic implant device is configured to elute the temporary carrier over about 40 days.

### CROSS-REFERENCE TO RELATED APPLICATION

This application is a continuation of U.S. Patent Application No. 63/549,423 filed on Feb. 2, 2024, entitled “FLUTED AND FENESTRATED INTRAMEDULLARY DEVICES,” the disclosure of which is incorporated herein in its entirety.

### TECHNICAL FIELD

The present disclosure relates generally to fluted and fenestrated intramedullary devices.

### BACKGROUND

Bone infection, also known as osteomyelitis, is an inflammatory process caused by pathogenic microorganisms, including bacteria, fungi, and mycobacteria. It may be acute or chronic and is acquired via open fractures, surgical interventions, or microorganisms spread from the bloodstream or surrounding soft tissue. Left untreated, the progression of the disease may be debilitating, requiring multiple surgical procedures to reach clinical resolution, or otherwise resulting in amputation or death in the most severe cases. Osteomyelitis patients can become chronically ill and suffer significant pain, disability, and high rates of amputation. In cases of osteomyelitis of the long bones (tibia, femur, humerus, etc.) the structural integrity of the bone may be diminished due to the infection or an underlying pathology such as osteoporosis. Surgical removal of the infected bone and soft tissues may be required.

A current standard of care for osteomyelitis patients is to remove the infected bone and soft tissues, thoroughly rinse the region with saline, insert a metal rod known as an intramedullary nail down the center of the medullary canal of the long bone, and administer systemic antibiotics. While non-invasive and inexpensive, systemic antibiotic therapy poses a risk of systemic toxicity and unwanted side effects. Systemic antibiotic therapy has reduced local efficacy due to limited blood flow near the implant interface and the medullary canal, and it increases the risk of antibiotic resistance and tolerance. Additionally, traditional fixation systems promote the development of adherent bacterial colonies, which form biofilms that are resistant to antibiotic treatment.

Currently, there are no FDA approved or cleared products intended for the simultaneous structural fixation of bone and delivery of local antibiotics for the treatment of osteomyelitis. Many surgeons construct off-label antibiotic cement-coated intramedullary nails (ACCIN) using bone cement and other miscellaneous items available to them in the operating room. Construction of these devices varies between surgeons but may take up to 45 minutes and typically involves the use of a silicone tube to facilitate the molding of antibiotic containing PMMA bone cement over a metal rod to provide reinforcement for partial weight bearing. Another strategy used by surgeons is mixing a resorbable bone void filler such as calcium sulfate with antibiotics and injecting it directly into the intramedullary canal before insertion of an intramedullary nail.

As a result, there is a long-felt, but unsolved need for devices for use in osteomyelitis patients that may combine the structural integrity of an intramedullary nail with the drug release properties of a resorbable carrier.

### BRIEF SUMMARY OF THE DISCLOSURE

One aspect of the present disclosure generally relates to an orthopedic implant device comprising an external surface having a plurality of fenestrations, and one or more radially distributed concave (i.e., recessed) longitudinal flutes oriented parallel to the longitudinal axis. In particular, the present disclosure relates an orthopedic implant device comprising longitudinal flutes including a bottom surface and a plurality of fenestrations through the bottom surface. Additionally, the present disclosure relates an orthopedic implant device including a central reservoir filled with a temporary carrier loaded with a therapeutic, connected to the plurality of fenestrations, wherein the orthopedic implant device is configured to elute the temporary carrier over about 40 days.

According to a first aspect, an orthopedic implant device comprising: a longitudinal axis, a diameter of about 7 mm to about 15 mm, a proximal end, a distal end, a device wall, a straight region comprising: one or more radially distributed concave longitudinal flutes oriented parallel to the longitudinal axis, each longitudinal flute comprising: a bottom surface, a flute length of about 100 mm to about 450 mm, a flute width of about 1 mm to about 3 mm, a flute depth of about 0.25 mm to about 1 mm, and a plurality of fenestrations through the bottom surface and extending into a central reservoir, each fenestration comprising a diameter of about 0.5 mm to about 3 mm, and at least one screw hole with a diameter of about 5 mm located near the distal end, wherein the device wall has a thickness of about 2 mm to about 4 mm and the central reservoir is configured for holding a volume of about 4 mL to about 9 mL of a temporary carrier loaded with at least one therapeutic.

According to a second aspect, the orthopedic implant device according to the first aspect or any other aspect, wherein the orthopedic implant device is an intramedullary nail.

According to a third aspect, the orthopedic implant device according to the second aspect or any other aspect, wherein the central reservoir is filled prior to intraoperative use with the temporary carrier loaded with at least one therapeutic.

According to a fourth aspect, the orthopedic implant device according to the second aspect or any other aspect, wherein the central reservoir is filled during intraoperative use with the temporary carrier loaded with at least one therapeutic.

According to a fifth aspect, the orthopedic implant device according to the second aspect or any other aspect, wherein the fenestrations are evenly spaced within each flute along the longitudinal axis.

According to a sixth aspect, the orthopedic implant device according to the fifth aspect or any other aspect, wherein the fenestrations are orthogonally oriented and offset in spacing.

According to a seventh aspect, the orthopedic implant device according to the sixth aspect or any other aspect, wherein the plurality of fenestrations comprises at least 7 fenestrations.

According to an eighth aspect, the orthopedic implant device according to the seventh aspect or any other aspect, wherein the central reservoir has a diameter of about 5 mm.

According to a ninth aspect, the orthopedic implant device according to the seventh aspect or any other aspect, wherein at a global force level of 750 N, the implant has a maximum local stress of about 350 to about 600 MPa.

According to a tenth aspect, the orthopedic implant device according to the nineth aspect or any other aspect, wherein the temporary carrier is calcium sulfate loaded with gentamicin and is configured for elution over about 40 days.

According to an eleventh aspect, the orthopedic implant device according to the nineth aspect or any other aspect, wherein the temporary carrier is calcium sulfate loaded with vancomycin and is configured for elution over about 40 days.

According to a twelfth aspect, the orthopedic implant device according to the nineth aspect or any other aspect, wherein the temporary carrier is calcium sulfate loaded with tobramycin and is configured for elution over about 40 days.

According to a thirteenth aspect, the orthopedic implant device according to the nineth aspect or any other aspect, wherein the temporary carrier is calcium sulfate loaded with gentamicin and vancomycin and is configured for elution over about 40 days.

According to a fourteenth aspect, the orthopedic implant device according to the nineth aspect or any other aspect, wherein the temporary carrier is calcium sulfate loaded with tobramycin and vancomycin and is configured for elution over about 40 days.

According to a fifteenth aspect, an orthopedic implant device comprising: a longitudinal axis, a diameter of about 7 mm to about 15 mm, one or more radially distributed concave longitudinal flutes oriented parallel to the longitudinal axis, each longitudinal flute comprising: a bottom surface, a width of about 1 mm to about 3 mm, a depth of about 0.25 mm to about 1 mm, and a plurality of fenestrations through the bottom surface and extending into a central reservoir, each fenestration comprising a diameter of about 0.5 mm to about 3 mm, and at least one screw hole, wherein the central reservoir is configured for holding a volume of about 4 mL to about 9 mL of a temporary carrier loaded with at least one therapeutic.

According to a sixteenth aspect, the orthopedic implant device according to the fifteenth aspect or any other aspect, wherein the device is an intramedullary nail.

According to a seventeenth aspect, the orthopedic implant device according to the fifteenth aspect or any other aspect, wherein the central reservoir is filled prior to intraoperative use with the temporary carrier loaded with at least one therapeutic.

According to an eighteenth aspect, the orthopedic implant device according to the fifteenth aspect or any other aspect, wherein the central reservoir is filled during intraoperative use with the temporary carrier loaded with at least one therapeutic.

According to a nineteenth aspect, the orthopedic implant device according to the sixteenth aspect or any other aspect, wherein each longitudinal flute is about 1 mm wide and about 1 mm deep.

According to a twentieth aspect, the orthopedic implant device according to the sixteenth aspect or any other aspect, wherein the plurality of fenestrations have a diameter of about 1 mm.

According to a twenty-first aspect, the orthopedic implant device according to the sixteenth aspect or any other aspect, wherein the plurality of fenestrations are evenly spaced within each flute along the longitudinal axis.

According to a twenty-second aspect, the orthopedic implant device according to the twenty-first aspect or any other aspect, wherein the plurality of fenestrations are orthogonally oriented and offset in spacing.

According to a twenty-third aspect, the orthopedic implant device according to the twenty-second aspect or any other aspect, wherein the plurality of fenestrations comprises at least 7 fenestrations.

According to a twenty-fourth aspect, the orthopedic implant device according to the twenty-third aspect or any other aspect, wherein the central reservoir has a diameter of about 5 mm.

According to a twenty-fifth aspect, the orthopedic implant device according to the twenty-fourth aspect or any other aspect, wherein at a global force level of 750 N, the implant has a maximum local stress of about 350 to about 600 MPa.

According to a twenty-sixth aspect, the orthopedic implant device according to the twenty-fifth aspect or any other aspect, wherein the temporary carrier is calcium sulfate loaded with at least gentamicin, vancomycin, or tobramycin and is eluted over about 40 days.

According to a twenty-seventh aspect, the orthopedic implant device according to the twenty-fifth aspect or any other aspect, wherein the temporary carrier is calcium sulfate loaded with gentamicin and vancomycin and is eluted over about 40 days.

According to a twenty-eighth aspect, the orthopedic implant device according to the twenty-fifth aspect or any other aspect, wherein the temporary carrier is calcium sulfate loaded with vancomycin and tobramycin and is eluted over about 40 days.

According to a twenty-nineth aspect, an orthopedic implant device comprising: a straight region comprising: an external surface having a plurality of fenestrations, one or more radially distributed concave longitudinal flutes oriented parallel to the longitudinal axis and comprising: a bottom surface, and a plurality of fenestrations through the bottom surface, and a central reservoir filled with a temporary carrier and connected to the plurality of fenestrations, wherein the orthopedic implant device is configured to elute the temporary carrier over about 40 days.

### BRIEF DESCRIPTION OF THE DRAWINGS

FIG. 1 is an exemplary fenestrated and fluted intramedullary nail placed within a tibia, according to one embodiment.

FIG. 2A is a perspective view of an exemplary fenestrated and fluted intramedullary device, according to one embodiment.

FIG. 2B is a cross-section view of the fenestrated and fluted intramedullary device of FIG. 2A, taken generally along the line 2-2 in FIG. 2A, according to one embodiment.

FIG. 2C is a side view of the fenestrated and fluted intramedullary device of FIG. 2A, according to one embodiment.

FIG. 2D is a cross-section view of the fenestrated and fluted intramedullary device of FIG. 2A, according to one embodiment.

FIG. 3A is perspective view of a fenestrated and fluted intramedullary device, according to one embodiment.

FIG. 3B is a cross-section view of the fenestrated and fluted intramedullary device of FIG. 3A, according to one embodiment.

FIGS. 4A-D illustrates varied flute dimensions in an intramedullary device, according to some embodiments.

FIG. 5 illustrates a method for conducting a Finite Element Analysis (FEA) analysis of a device, according to various embodiments.

FIGS. 6A and 6B illustrates a comparison of the impact dynamic fatigue performance of any exemplary device, according to various embodiments.

FIGS. 7A and 7B illustrates elution of gentamicin in fluted and fenestrated intramedullary nails compared to antibiotic cement coated intramedullary nails, according to one embodiment.

### DETAILED DESCRIPTION

Whether or not a term is capitalized is not considered definitive or limiting the meaning of a term. As used in this document, a capitalized term shall have the same meaning as an uncapitalized term is intended. However, the capitalization or lack thereof within the remainder of this document is not intended to be necessarily limiting unless the context clearly indicates that such limitation is intended.

Before any embodiments are described in detail, it is to be understood that the disclosure is not limited in its application to the details of construction and the arrangement of components set forth in the following description or illustrated in the following drawings, which is limited only by the claims that follow the present disclosure. The disclosure is capable of other embodiments, and of being practiced, or of being carried out, in various ways. Also, it is to be understood that the phraseology and terminology used herein are for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

The following description is presented to enable a person skilled in the art to make and use embodiments of the disclosure. Various modifications to the illustrated embodiments will be readily apparent to those skilled in the art, and the generic principles herein may be applied to other embodiments and applications without departing from embodiments of the disclosure. Thus, embodiments of the disclosure are not intended to be limited to embodiments shown but are to be accorded the widest scope consistent with the principles and features disclosed herein. The following detailed description is to be read with reference to the figures, in which like elements in different figures have like reference numerals. Skilled artisans will recognize the examples provided herein have many useful alternatives and fall within the scope of embodiments of the disclosure.

For the purpose of promoting an understanding of the principles of the present disclosure, reference will now be made to the embodiments illustrated in the drawings and specific language will be used to describe the same. It will, nevertheless, be understood that no limitation of the scope of the disclosure is thereby intended; any alterations and further modifications of the described or illustrated embodiments, and any further applications of the principles of the disclosure as illustrated therein are contemplated as would normally occur to one skilled in the art to which the disclosure relates. All limitations of scope should be determined in accordance with and as expressed in the claims.

### Overview

According to particular embodiments, the devices and systems herein are directed to intramedullary fixation systems comprising a sterile packaged and antibiotic-loaded orthopedic implant device which may be used to treat for example, but not limited to, long bone infections (e.g., in the tibia, femur, humerus, and hindfoot) as well as ensuring reliable mechanical stability. The orthopedic implant device may be an intramedullary nail. The intramedullary nail may be configured to treat the surrounding tissues via the gradual release of antibiotics (or another therapeutic). In some instances, antibiotics may be stored in a central reservoir within a body of the intramedullary nail. In some instances, the central reservoir may be filled with a temporary carrier (e.g., calcium sulfate bone void filler) loaded with at least one antibiotic. In one or more instances, the intramedullary nail may be cannulated and have fenestrations along the length. In some embodiments, the fenestrations in the intramedullary nail may act as release pores for the temporary carrier loaded with at least one antibiotic, which allows for a sustained therapeutic concentration of antibiotic to be released over time, preventing the colonization of microorganisms on the surface of the implant, and augmenting the efficacy of systemic antibiotics in the treatment of bone infections.

As will be understood from discussions herein, devices discussed herein may include, for example, nails, screws, pins, and other fixation mechanism or implants that elude a therapeutic treatment. In at least one example, the present disclosure relates to an implant that is patient-specific or used for defects other than intramedullary fixation (such, as for example, devices for total ankle, total knee, total hip, and/or total shoulder replacement).

According to particular embodiments, the devices and systems herein are directed to an orthopedic implant device comprising a straight region with an external surface having a plurality of fenestrations, one or more radially distributed concave longitudinal flutes oriented parallel to the longitudinal axis and comprising a bottom surface, and a plurality of fenestrations through the bottom surface, and a central reservoir filled with a temporary carrier and connected to the plurality of fenestrations, wherein the orthopedic implant device is configured to elute the temporary carrier over about 40 days.

In one or more embodiments, the orthopedic implant device comprises: a longitudinal axis, a diameter of about 7 mm to about 15 mm, a proximal end, a distal end, a device wall, a straight region comprising one or more radially distributed concave longitudinal flutes oriented parallel to the longitudinal axis, each longitudinal flute comprising a bottom surface, a flute length of about 100 mm to about 350 mm, a flute width of about 1 mm to about 3 mm, a flute depth of about 0.25 mm to about 1 mm, and a plurality of fenestrations through the bottom surface and extending into a central reservoir, each fenestration comprising a diameter of about 0.5 mm to about 3 mm; and the central reservoir comprising a temporary carrier loaded with at least one therapeutic, at least one screw hole with a diameter of about 5 mm located within the proximal bend region at the proximal end at least one screw hole with a diameter of about 5 mm located near the distal end, and wherein the device wall has a thickness of about 2 mm to about 4 mm and the central reservoir is configured for holding a volume of about 4 mL to about 9 mL of the temporary carrier. In some embodiments, the flute width is about 1 mm, and the flute depth is about 1 mm.

The orthopedic implant device may be an intramedullary nail, screw, or pin. The orthopedic implant device may include a plurality of fenestrations including at least 7 fenestrations. In some embodiments, the fenestrations may have a diameter of 1 mm. In various embodiments, the orthopedic implant device may include fenestrations that are orthogonally oriented and offset in spacing. In some embodiments, the fenestrations of the orthopedic implant device may be evenly spaced about 20 mm apart within each flute along the longitudinal axis. In various instances, the central reservoir of the implant as described may have a diameter of about 5 mm. Additionally and in some embodiments, upon application of a global force of 750 N, the orthopedic implant device may have a maximum local stress of about 350 to about 600 MPa. This force is intended to test durability of the orthopedic implant device for permanent implantation and weight bearing.

As will be understood, any suitable materials may be used in the implants described herein. In at least one embodiment, the orthopedic implant is constructed from surgical grade metal, stainless steel, titanium, cobalt chrome, or carbon reinforced polymer. In one or more embodiments, the temporary carrier in the above exemplary device is calcium sulfate. In one or more embodiments, the therapeutic agent in the above exemplary device is at least one antibiotic. In one or more embodiments, the therapeutic in the temporary carrier is at least gentamicin, vancomycin, or tobramycin and is eluted over about 40 days. In one or more embodiments, the therapeutic in the temporary carrier is gentamicin and tobramycin and is eluted over about 40 days. In one or more embodiments, the therapeutic in the temporary carrier is tobramycin and vancomycin and is eluted over about 40 days.

### Exemplary Device

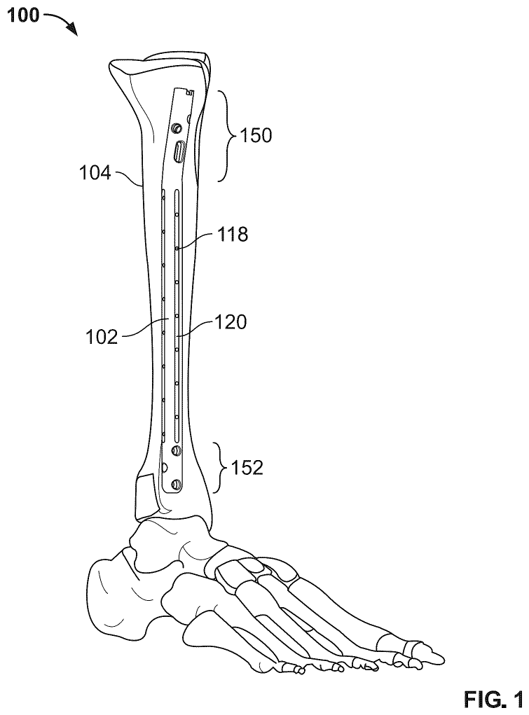

Turning now to FIG. 1, an exemplary orthopedic implant device 100 inserted into a bone is shown. In various embodiments, the orthopedic implant device 100 includes an implant body 102, a plurality of fenestrations 118, longitudinal flutes 120, a plurality of proximal locking screw holes 150, and a plurality of distal locking screw holes 152.

In at least one embodiment, an orthopedic implant device 100 may be inserted into a tibia bone 104. In alternative embodiments, the orthopedic implant device 100 may be inserted into a femur, humerus, hindfoot, metacarpal, phalanges, or any other suitable intramedullary canal or anatomic structure of a human or other animal. In some embodiments wherein the orthopedic implant device 100 may be inserted into a tibia bone 104, the orthopedic implant device 100 may optionally include a proximal bend. In other embodiments, the orthopedic implant device 100 may include only a straight region with no proximal bend.

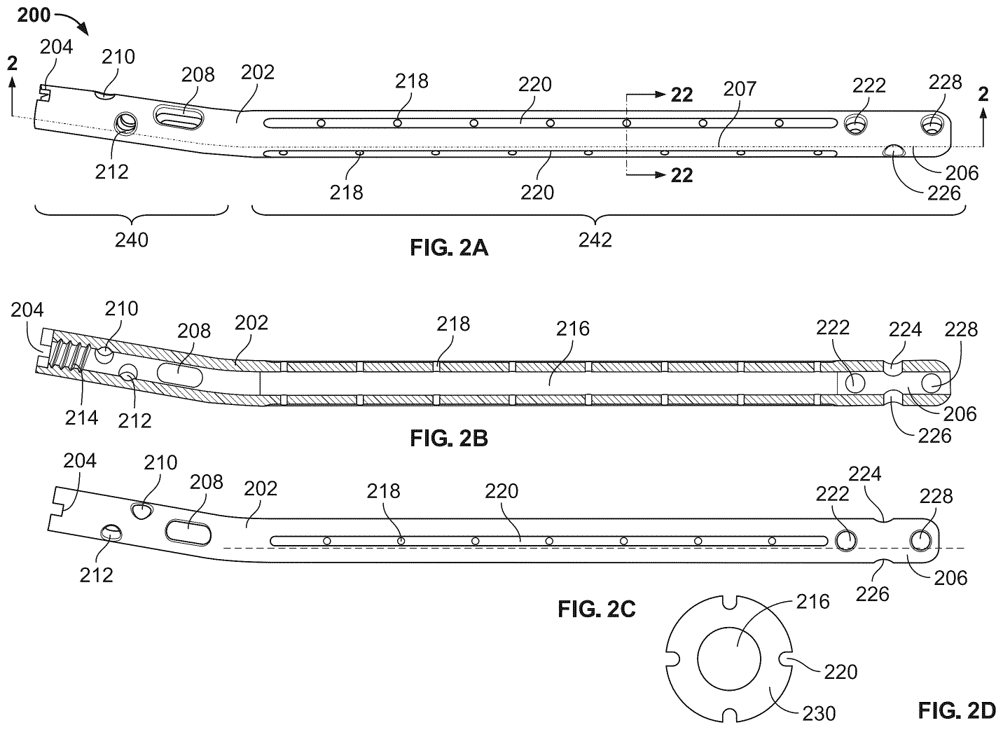

Referring now to FIGS. 2A-2D, various perspectives of an exemplary orthopedic device 200 are shown. FIGS. 2A and 2C show the orthopedic implant device 200 provided in the form of an implant body 202 with a proximal end 204, a distal end 206, a longitudinal axis 207, a first proximal screw hole 208, a second proximal screw hole 210, a third proximal locking screw hole 212, a plurality of fenestrations 218, one or more longitudinal flutes 220, a first distal screw hole 222, a second distal screw hole 224, a third distal screw hole 224, and a fourth distal screw hole 228. In various instances, the orthopedic implant device 200 may include any number of screw holes of any shape. In multiple embodiments, the orthopedic implant device 200 may be an intramedullary nail.

In some embodiments, the orthopedic implant device 200 optionally comprises a proximal bend 240 and a straight region 242. In multiple embodiments, the proximal bend 240 may comprise a 10° Herzog bend. In some instances, the proximal bend comprises a bend of at least 5 degrees, at least 10 degrees, at least 20 degrees, at least 30 degrees, or at least 45 degrees. In various embodiments, the orthopedic implant device 200 is a straight device with no proximal bend 240.

In some embodiments, the orthopedic implant device 200 comprises a length. In various embodiments, the orthopedic implant device 200 has a length of at least 10 mm, at least 50 mm, at least 100 mm, at least 200 mm, at least 300 mm, at least 400 mm, or at least 500 mm. In yet other embodiments, the orthopedic implant device 200 has a length of about 200 mm to about 500 mm, about 240 mm to about 490 mm, about 220 mm to about 450 mm, or about 240 mm to about 420 mm. In various instances, the orthopedic implant device 200 has a length of 200 mm to 500 mm, 240 mm to 490 mm, 220 mm to 450 mm, or 240 mm to 420 mm. In some embodiments, the orthopedic implant device 200 may be provided as an intramedullary nail with a length of 240 mm to 420 mm. Furthermore, in various embodiments, the orthopedic implant device 200 may comprise a very short, skinny intramedullary nail for insertion into bones in the hand or foot (e.g., the metacarpal, phalanges, or metatarsal).

In various instances, the orthopedic implant device 200 includes a diameter. The orthopedic implant device 200 may include a diameter of at least 1 mm, at least 5 mm, or at least 10 mm. In another embodiments, the orthopedic implant device 200 may include a diameter of about 7 mm to about 15 mm. In multiple instances, the orthopedic implant device 200 includes a diameter of about 9 mm, about 10 mm, or about 11.5 mm. In some embodiments, the orthopedic implant device 200 includes a diameter of 9 mm, 10 mm, or 11.5 mm. In yet another embodiment, the orthopedic implant device may include a uniform diameter throughout. In some instances, the orthopedic implant may comprise a non-uniform diameter throughout. For example, an embodiment may comprise a smaller diameter at a connection to the central reservoir and a wider diameter at the proximal 204 and distal 206 ends of the device.

In multiple embodiments, the orthopedic implant device 200 may include at least one screw hole. In some embodiments, the orthopedic implant device 200 may include at least one proximal screw hole 208, 210, 212. In another embodiment, the orthopedic implant device 200 may include at least one distal screw hole 222, 224, 226, 228. In some embodiments, the orthopedic implant device 200 may include at least one proximal screw hole 208, 210, 212 and at least one distal screw hole 222, 224, 226, 228. In multiple instances, the orthopedic implant device 200 may include more than one proximal screw hole 208, 210, 212. In various embodiments, the orthopedic implant device 200 may include at least three proximal screw holes 208, 210, 212. In some instances, the at least one proximal screw hole 208, 210, 212 may have a round shape including circular shapes or oval shapes. In some instances, the orthopedic implant device 200 comprises at least one proximal fastener hole.

In some instances, the at least one proximal screw hole 208, 210, 212 comprises a diameter of about 1 mm to about 5 mm. In multiple embodiments, the at least one proximal screw hole 208, 210, 212 may have a diameter of about 5 mm. In other embodiments, the at least one proximal screw hole 208, 210, 212 may have a diameter of at least 5 mm. In some embodiments, the at least one proximal screw hole 208, 210, 212 may have a diameter of 5 mm. In another embodiment, the orthopedic implant device 200 may include more than one distal screw hole 222, 224, 226, 228. In some instances, the orthopedic implant device 200 may include at least three distal screw holes 222, 224, 226, 228. In various embodiments, the orthopedic implant device 200 may include at least four distal screw holes 222, 224, 226, 228. In some instances, the at least one distal screw hole 222, 224, 226, 228 may have a round shape including circular shapes or oval shapes. In some instances, the orthopedic implant device 200 comprises at least one distal fastener hole.

In another embodiment, the at least one distal screw hole 222, 224, 226, 228 comprises a diameter of about 1 mm to about 5 mm. In multiple embodiments, the at least one distal screw hole 222, 224, 226, 228 may have a diameter of about 5 mm. In some embodiments, the at least one distal screw hole 222, 224, 226, 228 may have a diameter of at least 5 mm. In various embodiments, the at least one distal screw hole 222, 224, 226, 228 may have a diameter of 5 mm.

Furthermore, in some embodiments, the orthopedic implant device 200 may comprise one or more flutes 220. The one or more flutes 220 may include a bottom surface. The one or more flutes 220 may be configured as U-shaped, V-shaped, square shaped, shaped in a slit configuration, or shaped in a non-symmetrical shape. In various embodiments, the one or more flutes 220 may comprise concave shaped flutes 220. Additionally, in some embodiments, the orthopedic implant device 200 may comprise more than one flute 220 wherein the flutes are the same shape. In other embodiments, the orthopedic implant device 200 may comprise more than one flute 220 wherein the flutes are different shapes. In some instances, the one or more flutes 220 may be longitudinal. In some embodiments, the one or more flutes 220 may be parallel to a longitudinal axis 207. In other embodiments, the one or more flutes 220 are not longitudinal. In yet another instance, the one or more flutes 220 may not be parallel to the longitudinal axis 207. In multiple embodiments, the flutes 220 may be radially distributed. In other instances, the flutes 220 may not be radially distributed. In some embodiments, the flutes 220 may extend concentrically around the longitudinal axis 207.

In multiple instances, the orthopedic implant device 200 may include any number of flutes 220. The distribution of the any number of flutes 220 may have various configurations. In some embodiments, the orthopedic implant device 200 may include at least 4 radially distributed longitudinal flutes 220. In other embodiments, the longitudinal flutes 220 are not radially distributed. In multiple embodiments, the flutes 220 may be positioned on various directions of the orthopedic implant device 200 including an anterior, a posterior, a medial, and a lateral direction. In some instances, the orthopedic implant device 200 may include one or more flutes 220 where all the flutes 220 are positioned on one direction. In yet another embodiment, the flutes 220 may be positioned on two directions across from each other (e.g., on anterior and posterior sides). In another instance, the flutes 220 may be positioned on only 3 of the directions (e.g., on anterior, lateral, and posterior sides).

In various embodiments, the longitudinal flutes 220 may include a flute length. In some instances, the longitudinal flutes 220 may include any flute length. In some embodiments, each longitudinal flute 220 has a flute length of at least 10 mm, at least 50 mm, at least 100 mm, at least 200 mm, at least 300 mm, at least 400 mm, at least 450 mm, or at least 500 mm. In multiple embodiments, each longitudinal flute 220 has a flute length of about 100 mm to 350 mm. In various embodiments, each longitudinal flute 220 has a flute length of about 100 mm to 450 mm. In multiple instances, each longitudinal flute 220 has a flute length of about 250 mm to 450 mm. In other embodiments, each longitudinal flute 220 has a flute length of about 300 mm to 450 mm. In some instances, the length of the longitudinal flutes 220 may be equivalent for each longitudinal flute 220. In other instances, the length of the longitudinal flutes 220 may vary for each longitudinal flute 220. For example, in some embodiments, the longitudinal flutes 220 facing the anterior (A) and posterior (P) directions may be longer than the longitudinal flutes 220 facing the medial (M) and lateral (L) directions as show in Table 1 below.

TABLE 1

| | A/P Flute Length | M/L Flute Length |
|---|---|---|
| Nail Length (mm) | (mm) | mm |
| 240 | 150 | 130 |
| 260 | 170 | 150 |
| 280 | 190 | 170 |
| 300 | 210 | 190 |
| 320 | 230 | 210 |
| 340 | 250 | 230 |
| 360 | 270 | 250 |
| 380 | 290 | 270 |
| 400 | 310 | 290 |
| 420 | 330 | 310 |

In various embodiments, the longitudinal flutes 220 facing the anterior (A) and posterior (P) directions may have a flute length of at least 10 mm, at least 50 mm, at least 100 mm, at least 200 mm, at least 300 mm, at least 400 mm, or at least 450 mm, or at least 500 mm. In some embodiments, the longitudinal flutes 220 facing the anterior (A) and posterior (P) directions may have a flute length of about 100 mm to about 350 mm. In multiple embodiments, the longitudinal flutes 220 facing the anterior (A) and posterior (P) directions may have a flute length of about 150 mm to about 290 mm. In yet another embodiment, the longitudinal flutes 220 facing the medial (M) and lateral (L) directions may have a flute length of about 100 mm to about 350 mm. In some instances, the longitudinal flutes 220 facing the medial (M) and lateral (L) directions may have a flute length of about 130 mm to about 310 mm. In various embodiments, the longitudinal flutes 220 facing the medial (M) and lateral (L) directions may have a flute length that is greater than the length of the than the longitudinal flutes 220 facing the anterior (A) and posterior (P) directions by at least 1 mm, at least 5 mm, at least 10 mm, at least 20 mm, at least 30 mm, or at least 40 mm. In various instances, the longitudinal flutes 220 facing the anterior (A) and posterior (P) directions may have a flute length that is greater than the length of the than the longitudinal flutes 220 facing the medial (M) and lateral (L) by at least 1 mm, at least 5 mm, at least 10 mm, at least 20 mm, at least 30 mm, or at least 40 mm.

In some embodiments, the longitudinal flutes 220 may include a flute width. In various embodiments, each longitudinal flute 220 may have a flute width of about 0.25 mm to about 5 mm. In other embodiments, each longitudinal flute 220 may have a flute width of about 0.5 mm to about 5 mm. In multiple embodiments, each longitudinal flute 220 may have a flute width of about 1 mm to about 3 mm. In yet another embodiment, each longitudinal flute 220 may have a flute width of about 1 mm. In some instances, each longitudinal flute 220 may have a flute width of 1 mm.

In various embodiments, the longitudinal flutes 220 may include a flute depth. In some instances, the longitudinal flutes 220 may include any flute depth. In some embodiments, each longitudinal flute 220 may have a flute depth of 0.25 mm to about 5 mm. In another embodiment, each longitudinal flute 220 may have a flute depth of 0.25 mm to about 5 mm. In various embodiments, each longitudinal flute 220 may have a flute depth of about 0.25 mm to about 1 mm. In another instance, each longitudinal flute 220 may have a flute depth of about 1 mm. In some embodiments, each longitudinal flute 220 may have a flute depth of 1 mm.

In addition, in multiple embodiments, the orthopedic implant device 200 may include a plurality of fenestrations 218. In some embodiments, the implant device 200 comprises a plurality of fenestrations 218 wherein the fenestrations are located through the bottom surface of a longitudinal flute 220. In various instances, the implant device 200 comprises any number of fenestrations 218 within each flute 220. In yet other embodiments, the implant device 200 can comprise 1 to 100 fenestrations 218 per flute 220.

In another embodiment, the implant device 200 comprises about 1 to about 20 fenestrations 218 per flute 220. In some instances, the implant device 200 comprises 1 to 20 fenestrations 218 per flute 220. In multiple embodiments, the plurality of fenestrations 218 includes about 1 to about 17 fenestrations within the longitudinal flute 220. In some embodiments, the plurality of fenestrations 218 includes 7 to 17 fenestrations within the longitudinal flute 220. Furthermore, in another embodiment, the plurality of fenestrations 218 includes at least 4, at least 5, at least 6, at least 7, at least 8, at least 9, at least 10, at least 11, at least 12, at least 13, or at least 14 fenestrations within the longitudinal flute 220. In multiple embodiments, the plurality of fenestrations 218 includes at least 7 fenestrations within the longitudinal flute 220.

In various embodiments, the plurality of fenestrations 218 may have a shape. In some instances, the plurality of fenestrations 218 may have a round or non-round shape. Round shapes may include circular shapes or oval shapes. Non-round shapes may include slots, X shaped, square, or slit shapes. In some instances, the fenestrations 218 may be shaped as a slit through the bottom of the flute 220. In some embodiments, the plurality of fenestrations 218 may include chamfers.

In multiple instances, the plurality of fenestrations 218 comprise a diameter. In multiple embodiments, the plurality of fenestrations 218 may have a diameter of about 0.25 mm to about 5 mm. In some embodiments, the plurality of fenestrations 218 may have a diameter of about 0.5 mm to about 3 mm. In another embodiment, the plurality of fenestrations 218 may have a diameter of about 1 mm. In yet another embodiment, the plurality of fenestrations 218 may have a diameter of 1 mm.

In multiple instances, the orthopedic implant device 200 comprises a plurality of fenestrations 218 that are distributed and oriented in relation to each other. In various embodiments, the plurality of fenestrations 218 may be orthogonally oriented and offset in spacing to one another. In another embodiment, the fenestrations 218 are not orthogonally oriented. In yet another embodiment, the fenestrations 218 are not offset from each other. In various instances, the plurality of fenestrations 218 are distributed evenly within a flute 220. In other instances, the plurality of fenestrations 218 are distributed unevenly within a flute 220. For example, the fenestrations 218 may be distributed in a manner where a greater number of fenestrations are present at one region of the implant device 200 than another region of the implant device 200 in order to direct therapeutic treatment. The plurality of fenestrations 218 may be concentrated in the implant device 200 based on area of greatest therapeutic need. The orthopedic implant device 200 may have a greater density of fenestrations 218 (or a single fenestration) in one or more regions of the implant device 200 that correspond with treating a local infection in the bone upon insertion (e.g., more fenestrations or a greater surface area of fenestrations near a proximal or distal end of an implant).

The plurality of fenestrations 218 may be oriented along the longitudinal axis 207 at a distance from each other. In various embodiments, within the plurality of fenestrations 218, each fenestration 218 may be spaced evenly from each other along the longitudinal axis 207. In various embodiments, within the plurality of fenestrations 218, each fenestration 218 may be located about 2 to about 30 mm apart from each other along the longitudinal axis 207. In various embodiments, within the plurality of fenestrations 218, each fenestration 218 may be located 2 to 30 mm apart from each other along the longitudinal axis 207. In some embodiments, each fenestration 218 may be located about 10 to about 20 mm apart from each other along the longitudinal axis 207. In multiple embodiments, each fenestration 218 may be located about 20 mm apart from each other along the longitudinal axis 207. In another embodiment, each fenestration 218 may be located 20 mm apart from each other along the longitudinal axis 207.

FIG. 2B shows a cross section view of the orthopedic implant device 200 through FIG. 2A at A-A. In various embodiments, the orthopedic implant device 200 may include a threaded portion 214 for connection to an insertion tool or another fixation device. In some embodiments, the orthopedic implant device 200 may include a central reservoir 216. The central reservoir 216 may be filled with a temporary carrier loaded with a volume of at least one therapeutic. In some instances, the central reservoir 216 is configured for holding a volume of about 1 mL to about 15 mL of the temporary carrier. In some instances, the central reservoir 216 is configured for holding a volume of about 4 mL to about 9 mL of the temporary carrier. In other instances, the central reservoir 216 is configured for holding a volume of 4 mL to 9 mL of the temporary carrier. In some embodiments, the temporary carrier may be a resorbable bone void filler. In some instances, the temporary carrier may be configured as beads. In various embodiments, the temporary carrier may include a calcium salt. In another embodiment, the temporary carrier may include calcium sulfate. In yet another embodiment, the temporary carrier may include calcium phosphate. In some instances, the temporary carrier may include hydroxyapatite. In another instance, the temporary carrier may include calcium sulfate, tricalcium phosphate, and hydroxyapatite. In some embodiments, the temporary carrier may include calcium sulfate hemihydrate. In some instances, the temporary carrier is biodegradable.

In multiple embodiments, the temporary carrier may be loaded with at least one therapeutic. The at least one therapeutic may include antibiotics, antifungals, analgesics, and chemotherapeutics. The therapeutic may include any antibiotic, any antifungal, any analgesic, and any chemotherapeutic. In some instances, the temporary carrier may be loaded with an amount of therapeutic of at least 0.01% wt/wt, 5% wt/wt, at least 10% wt/wt, at least 20% wt/wt, or at least 30% wt/wt.

In some embodiments, the temporary carrier may be loaded with at least one therapeutic comprising antibiotics. In some embodiments, the temporary carrier may be loaded with at least one therapeutic comprising at least gentamicin, vancomycin, or tobramycin. In another instance, the temporary carrier may be loaded with at least one therapeutic comprising the therapeutic including at least gentamicin, vancomycin, or tobramycin wherein the amount of therapeutic is at least 5% wt/wt, at least 10% wt/wt, at least 20% wt/wt, or at least 30% wt/wt.

In some instances, the central reservoir 216 of the orthopedic implant device 200 may be filled any time prior to intraoperative use. In various instances, the central reservoir 216 is filled prior to intraoperative use with the temporary carrier loaded with at least one therapeutic. According to particular embodiments, the central reservoir 216 of the orthopedic implant device 200 may be filled upon manufacturing and assembly, before being received at a hospital, ambulatory surgery center (ASC), or another operative environment. In various instances, the central reservoir 216 of the orthopedic implant device 200 may be filled at least 12 hours prior to implantation. In another embodiment, the central reservoir 216 of the orthopedic implant device 200 may be filled at least 1 minute, at least 10 minutes, at least 30 minutes, at least 1 hour, or at least 2 hours prior to implantation. In yet another embodiment, the central reservoir 216 of the orthopedic implant device 200 may be filled during intraoperative use. In other embodiments, the central reservoir 216 is filled during intraoperative use with the temporary carrier loaded with at least one therapeutic. In various instances, the central reservoir 216 of the orthopedic implant device 200 may be filled after the device is implanted in the patient.

In multiple embodiments, the central reservoir 216 of the orthopedic implant device 200 may be filled intraoperatively with antibiotic beads. In some instances, the antibiotic beads can be formed intraoperatively and filled into the central reservoir 216 of the orthopedic implant device 200. In other instances, the beads comprise other therapeutic agents instead of or in addition to antibiotics. The use of beads to fill the central reservoir can increase the surface area and thereby increase the elution activity. As will be understood, “beads” may be any suitable shape, including substantially spherical, conical, cuboid, semi-spherical (e.g., a half, quarter, or other portion of a sphere). In some embodiments, the antibiotic or therapeutic may be in the form of randomly shaped grounds, powder, or other suitable material construct.

Additionally, the therapeutic may elute from the central reservoir 216 into the surrounding bone and tissue. In some embodiments, the therapeutic may be eluted from the central reservoir 216 over about 1 day to 365 days. In other embodiments, the therapeutic may be eluted from the central reservoir 216 over about 1 hour to about 6 hours, over about 6 hours to about 12 hours, or over about 12 hours to about 24 hours. In various instances, the therapeutic may be eluted from the central reservoir 216 over at least 2 months, at least 4 months, at least 6 months, at least 8 months, at least 10 months, or at least a year. In some embodiments, the therapeutic may be eluted from the central reservoir 216 over about 20 days to 60 days. In some embodiments, the therapeutic may be eluted from the central reservoir 216 over about 40 days. In some embodiments, the therapeutic may be eluted from the central reservoir 216 over at least 40 days.

In some instances, the temporary carrier may be calcium sulfate loaded with gentamicin and vancomycin and is eluted from the central reservoir 216 over about 1 day to 365 days. In other embodiments, the temporary carrier may be calcium sulfate loaded with gentamicin and vancomycin and is eluted from the central reservoir 216 over about 1 hour to about 6 hours, over about 6 hours to about 12 hours, or over about 12 hours to about 24 hours. In various instances, the temporary carrier may be calcium sulfate loaded with gentamicin and vancomycin and is eluted from the central reservoir 216 over at least 2 months, at least 4 months, at least 6 months, at least 8 months, at least 10 months, or at least a year. In some instances, the temporary carrier may be calcium sulfate loaded with gentamicin and vancomycin and is eluted from the central reservoir 216 over about 20 days to about 60 days. In some instances, the temporary carrier may be calcium sulfate loaded with gentamicin and vancomycin and is eluted from the central reservoir 216 over about 40 days. In some instances, the temporary carrier may be calcium sulfate loaded with gentamicin and vancomycin and is eluted from the central reservoir 216 over at least 40 days.

In some embodiments, the temporary carrier may be calcium sulfate loaded with gentamicin and tobramycin and is eluted from the central reservoir 216 over about 1 day to 365 days. In other embodiments, the temporary carrier may be calcium sulfate loaded with gentamicin and tobramycin and is eluted from the central reservoir 216 over about 1 hour to about 6 hours, over about 6 hours to about 12 hours, or over about 12 hours to about 24 hours. In various instances, the temporary carrier may be calcium sulfate loaded with gentamicin and tobramycin and is eluted from the central reservoir 216 over at least 2 months, at least 4 months, at least 6 months, at least 8 months, at least 10 months, or at least a year. In at least one instance, the temporary carrier may be calcium sulfate loaded with gentamicin and tobramycin and is eluted from the central reservoir 216 over about 30 days to about 60 days. In some instances, the temporary carrier may be calcium sulfate loaded with gentamicin and tobramycin and is eluted from the central reservoir 216 over about 40 days. In another embodiment, the temporary carrier may be calcium sulfate loaded with gentamicin and tobramycin and is eluted from the central reservoir 216 over at least 40 days.

In some instances, the temporary carrier may be calcium sulfate loaded with vancomycin and tobramycin and is eluted from the central reservoir 216 over about 30 days to about 60 days. In another embodiment, the temporary carrier may be calcium sulfate loaded with vancomycin and tobramycin and is eluted from the central reservoir 216 over about 40 days. In various instances, the temporary carrier may be calcium sulfate loaded with vancomycin and tobramycin and is eluted from the central reservoir 216 over at least 40 days.

FIG. 2D shows a cross section view of the orthopedic implant device 200 through FIG. 2A at B-B. In some embodiments, the orthopedic implant device 200 also includes the central reservoir 216, a device wall 230, and longitudinal flutes 220 oriented parallel to the longitudinal axis 207 (See FIG. 2C). In various embodiments, the orthopedic implant device 200 incudes the central reservoir 216 connected to the plurality of fenestrations 218. In another instance, the central reservoir 216 may have a diameter of at least 0.5 mm, at least 1 mm, at least 3 mm, or at least 5 mm. In some embodiments, the central reservoir 216 may have a diameter of about 1 mm to about 5 mm. In various instances, the longitudinal cannulation of the nail has a diameter of about 5 mm. In one embodiment, the longitudinal cannulation of the nail has a diameter of 5 mm. Furthermore, in multiple embodiments, the central reservoir may comprise a longitudinal cannulation. In some instances, the central reservoir may be capped. In alternative embodiments, the central reservoir may be plugged by a biodegradable substance.

In various embodiments, the orthopedic implant device 200 includes one or more flutes. As an example, the orthopedic implant device 200 may include concave longitudinal flutes 220 equally spaced apart along the longitudinal axis 207. In one or more embodiments, the orthopedic implant device 200 may include at least 4 concave longitudinal flutes 220 oriented orthogonally to each other. In an embodiment, the orthopedic implant device 200 includes at least 4 concave longitudinal flutes 220 which may be evenly placed and oriented at a 90-degree angle to each other. According to particular embodiments, the orthopedic implant device 200 includes one or more flutes or more than four flutes.

In various embodiments, the orthopedic implant device 200 includes the device wall 230. In some instances, the device wall 230 has a thickness of at least 0.25 mm, at least 0.5 mm, at least 1 mm, at least 2 mm, at least 3 mm, at least 4 mm, or at least 5 mm. In some embodiments, the device wall 230 has a thickness of about 2 mm to about 4 mm. In another instance, the device wall 230 has a thickness of 2 mm to 4 mm. In multiple embodiments, the device wall 230 is smooth. In alternative embodiments the device wall 230 is rough. In some instances, the device wall 230 can be coated with a treatment or therapeutic to inhibit microbial growth.

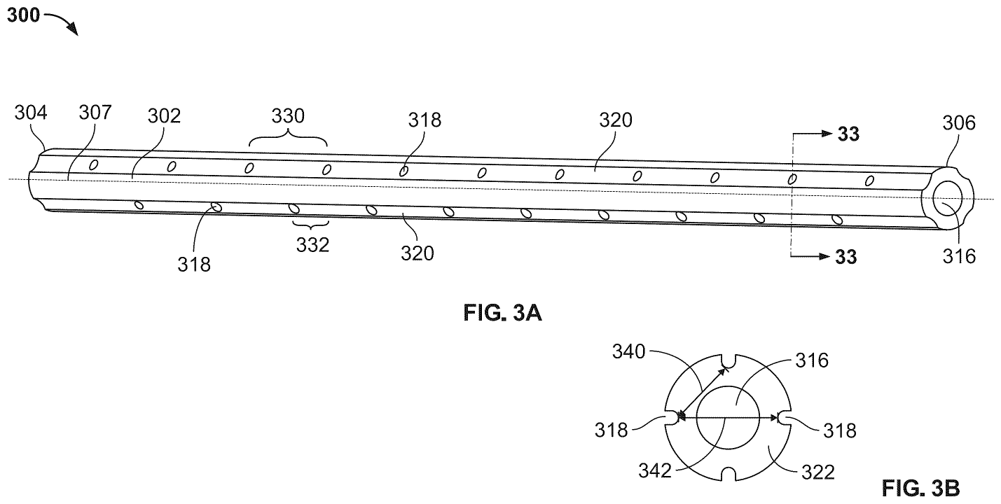

Turning to FIGS. 3A and 3B, various perspectives of an exemplary orthopedic device 300 are shown. FIGS. 3A and 3B show the orthopedic implant device 300 including an implant body 302, a proximal end 304, a distal end 306, a longitudinal axis 307, a central reservoir 316, a plurality of fenestrations 318, and one or more radially distributed concave longitudinal flutes 320. In some embodiments, the orthopedic implant device 300 is straight or generally straight. In one or more embodiments, the orthopedic implant device 300 can have at least one screw hole. In various embodiments, the orthopedic implant device 300 may be an intramedullary nail.

In various embodiments, the orthopedic implant device 300 has a substantially similar length, diameter, and device wall as the implant device 200 shown in FIG. 2 and described herein.

In multiple embodiments, the orthopedic implant device 300 may include at least one screw hole, at least one proximal screw hole, and/or at least one distal screw hole as the implant device 200 shown in FIG. 2 and described herein.

In various embodiments, the orthopedic implant device 300 may have a central reservoir 316 substantially similar to the central reservoir of implant device 200 shown in FIG. 2 and described herein. In some embodiments, the orthopedic implant device 300 incudes the central reservoir 316 connected to the plurality of fenestrations 318. The central reservoir may be filled with a temporary carrier loaded with a therapeutic as described herein.

In multiple embodiments, the orthopedic implant device 300 may include a plurality of fenestrations 318 as the implant device 200 shown in FIG. 2 and described herein.

The plurality of fenestrations 318 may be oriented along the longitudinal axis 307 at a distance from each other. In various embodiments, the plurality of fenestrations 318 may be located through the bottom of the radially distributed concave longitudinal flutes 320. In some embodiments, within the plurality of fenestrations 318, each fenestration 318 may be located a first distance 330 apart from each other along the longitudinal axis. In various embodiments, the first distance 330 is equivalent between all fenestrations 318 within the same longitudinal flute 320. In some embodiments, the first distance 330 is evenly spaced between all fenestrations 318 within the same longitudinal flute 320. In some embodiments, the first distance 330 may be about 5 to about 30 mm. In another embodiment, the first distance 330 may be about 10 to about 20 mm. In yet another embodiment, the first distance 330 may be about 20 mm. In some instances, the first distance 330 may be 20 mm. In various embodiments, within the plurality of fenestrations 318, each fenestration 318 may be located a second distance 332 offset from each other along the longitudinal axis. In some embodiments, the second distance 332 is equivalent between all fenestrations 318 within the same longitudinal flute 320. In multiple instances, the second distance 332 may be about 5 to about 30 mm. In another instance, the second distance 332 may be about 10 to about 20 mm. In some embodiments, the second distance 332 may be about 10 mm. In some embodiments, the second distance 332 may be 10 mm.

FIG. 3B shows a cross section view of the orthopedic implant device 300 through FIG. 3A at C-C. In some embodiments, the orthopedic implant device 300 includes the central reservoir 316, a device wall 322, and the concave longitudinal flutes 320 oriented parallel to the longitudinal axis 307 (See FIG. 3A). In the exemplary orthopedic implant device 300, the device 300 may include one or more radially distributed concave longitudinal flutes 320 wherein the longitudinal flutes 320 may be oriented parallel to the longitudinal axis 307. In various embodiments, the one or more radially distributed concave longitudinal flutes 320 may include a shape, a bottom surface, a flute length, a flute width, and a flute depth as described in FIG. 2 describing the orthopedic implant device 200.

In some embodiments, the orthopedic implant device 300 includes concave longitudinal flutes 320 oriented in relation to another. In an embodiment, the orthopedic implant device 300 includes concave longitudinal flutes 320 equally spaced apart along the longitudinal axis 307. In some embodiments, the orthopedic implant device 300 may include at least 4 concave longitudinal flutes 320 oriented orthogonally to each other. In some embodiments, the orthopedic implant device 300 includes at least 4 concave longitudinal flutes 320 which may be evenly placed and oriented at a 90-degree angle to each other. In alternative embodiments, the orthopedic implant device 300 may include any number of flutes 320. Referring to FIG. 3B, in some embodiments, the longitudinal flutes 320 are located across a third distance 340 from each other. In various instances, the longitudinal flutes 320 are located across a fourth distance 342 from each other. In yet another embodiment, the distance between each longitudinal flute 320 varies with device diameter. For example, in an embodiment, if the nail has a diameter of 9 mm and the longitudinal flutes 320 have a flute depth of 1 mm, the longitudinal flutes 320, the third distance 340 would be 4.95 mm. In the same example, the fourth distance 342 would be 7 mm.

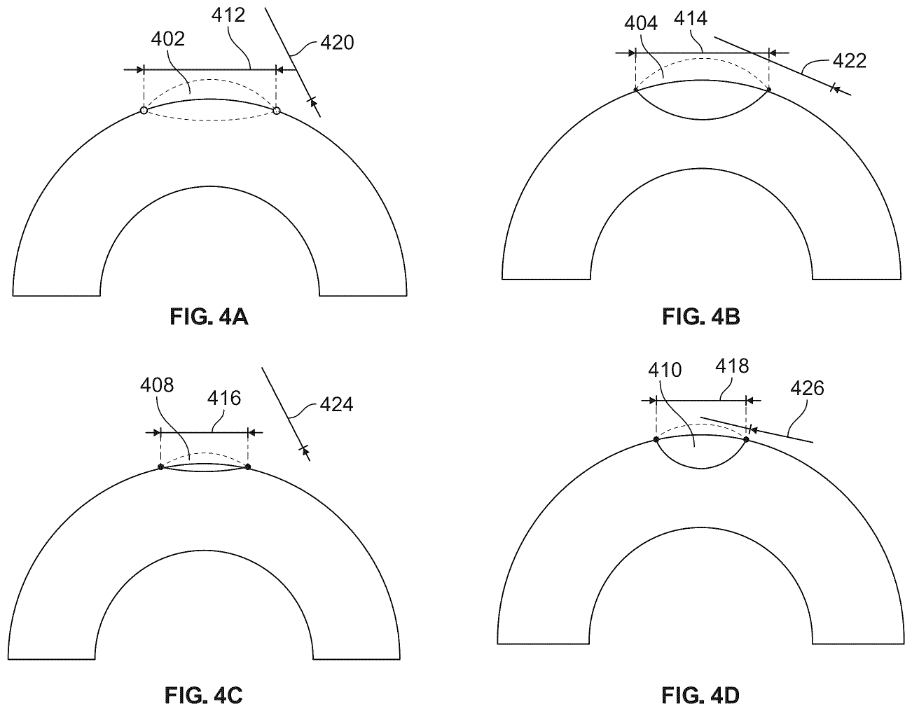

Turning to FIGS. 4A-4B, various embodiments of the longitudinal flutes 120, 220, 320 which may be incorporated into the orthopedic implant device 100, 200, 300 are shown. FIGS. 4A-4B illustrate varying dimensions of the longitudinal flutes 120, 220, 320 that may be incorporated. FIG. 4A depicts one embodiment of a longitudinal flute 120, 220, 320 provided with a flute dimension 402 comprising a flute width 412 and a flute radius of the cut 420. In an embodiment, the flute dimension 402 comprises a flute width 412 of 3 mm and a flute radius of the cut 420 of 5 mm.

FIG. 4B depicts an embodiment of a longitudinal flute 120, 220, 320 provided with a flute dimension 404 comprising a flute width 414 and a flute radius of the cut 422. In an embodiment, the flute dimension 404 comprises a flute width 414 of 3 mm and the flute radius of the cut 422 of 2 mm.

FIG. 4C depicts an embodiment of a longitudinal flute 120, 220, 320 provided with a flute dimension 408 comprising a flute width 416 and a flute radius of the cut 424. In an embodiment, the flute dimension 408 comprises a flute width 416 of 2 mm and the flute radius of the cut 424 of 5 mm.

FIG. 4D depicts an embodiment of a longitudinal flute 120, 220, 320 provided with a flute dimension 410 comprising a flute width 418 and a flute radius of the cut 426. In an embodiment, the flute dimension 410 comprises a flute width 418 of 2 mm and the flute radius of the cut 426 of 1.1 mm.

In other embodiments of longitudinal flutes 120, 220, 320 which may be incorporated into the orthopedic implant device 100, 200, 300 the flute width may comprise a width of at least 0.25 mm, at least 0.5 mm, at least 1 mm, at least 2 mm, at least 3 mm, at least 4 mm, or at least 5 mm. In yet another embodiment of longitudinal flutes 120, 220, 320 which may be incorporated into the orthopedic implant device 100, 200, 300 the flute radius of the cut may comprise a radius of the cut of at least 0.25 mm, at least 0.5 mm, at least 1 mm, at least 2 mm, at least 3 mm, at least 4 mm, or at least 5 mm.

### Exemplary Mechanical Data

One unique challenge is that fenestrations within an orthopedic implant device may create local stress concentrations, which may lower the fatigue bending strength of a device and may result in implant failure. In order to overcome this challenge, the various embodiments of the orthopedic implant device 100, 200, 300 were tested via a Finite Element Analysis (FEA) to determine embodiments of the orthopedic implant device 100, 200, 300 that mitigate the impact of the fenestrations on the mechanical performance of the orthopedic implant device 100, 200, 300. Surprisingly, the experimental mechanical data shows that devices with thin and deep longitudinal flutes and fenestrations mitigate the impact of the fenestrations on the mechanical performance of the orthopedic implant device 100, 200, 300.

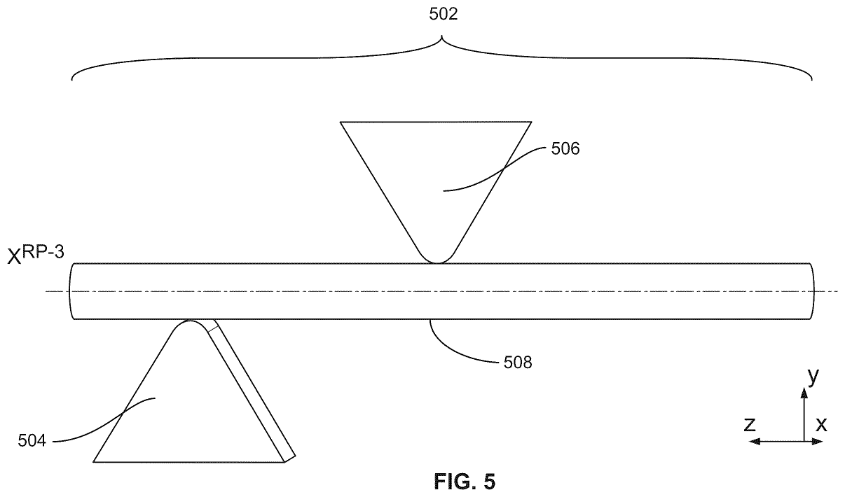

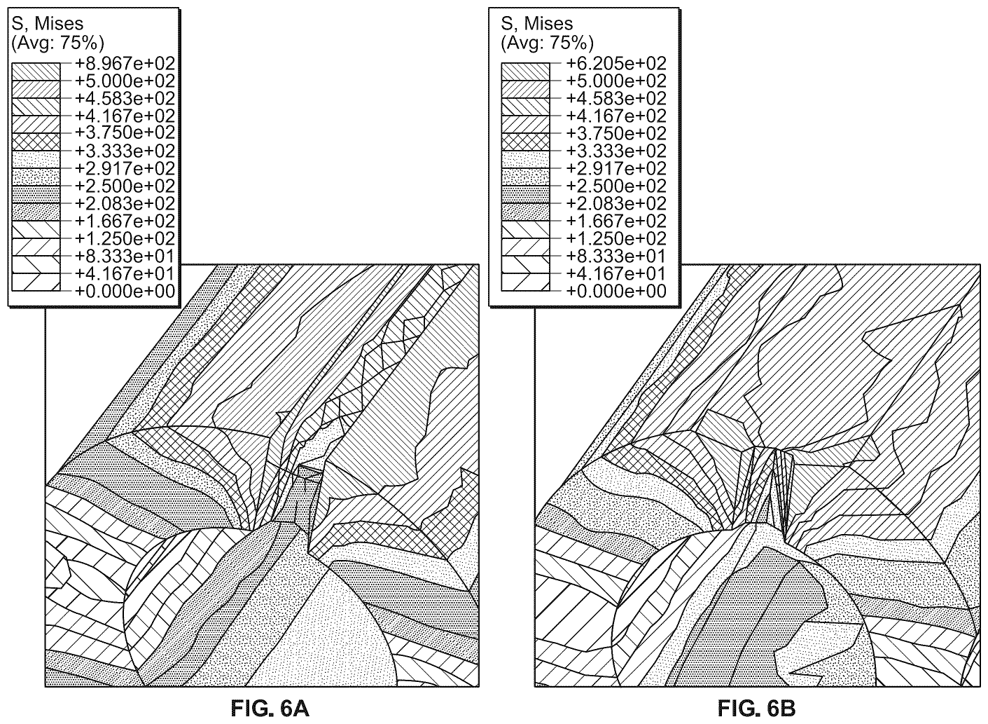

FIGS. 5 and 6 illustrate methods of analysis and mechanical properties of the various embodiments of the orthopedic implant device 100, 200, 300.

FIG. 5 illustrates one method for setting up a Finite Element Analysis (FEA) analysis of an exemplary device. The FEA method may be utilized in the geometric design of test specimens for use in 4-point plane beam bending fatigue test, allowing for accurate predictions of bending stresses, as well as reliable predictions of the location of fatigue crack initiation.

In various embodiments, in order to assess the static bending strength of various embodiments of the orthopedic implant device 100, 200, 300, the FEA method above was used. The FEA method included a simulated 4-point test using a cut made at a midpoint 508 of a span length 502 of the device models to simplify the computational demands of the simulation using a symmetry enforcement. In the simulation, a top roller 504 and a bottom roller 506 were modeled as a discrete rigid part and the orthopedic implant device 100, 200, 300 was meshed with linear tetrahedral elements, and the rollers 504, 506 were meshed with linear triangular elements.

In various embodiments, mesh sensitivity analysis of the orthopedic implant device 100, 200, 300, was performed to ensure the size of the mesh did not contribute to the results of the simulation for bending strength.

In some embodiments, the assessment of static bending strength using the 4-point test simulation included boundary conditions and loading conditions selected to replicate physical benchtop setups per ASTM 1264.15. In various instances, the setup of the 4-point test simulation was loaded under a linear displacement rate at the center point between rollers 504, 506 with the span length 502 of 114 mm as shown in FIG. 5.

In the assessment of static bending strength using the 4-point test simulation, the bottom roller 506 was fixed with an encastre condition, and a displacement was applied to the top roller 504 in the downward direction, while all other degrees of freedom were fixed. In the assessment of static bending strength using the 4-point test simulation, symmetry conditions about the z-axis were defined for the nodes on the surface of the midpoint of the device. In addition, rotation around the z-axis was also restricted for these nodes. In some embodiments, the output of this simulation is a force-displacement curve, extracted from the displacement and reaction force of the top roller 504. In various instances, the maximum local stress outside of the roller contacting region was recorded as a proxy for bending fatigue performance. In other embodiments, the FEA method as described herein was used to determine crosshead displacement, or the movement of the crosshead in the y direction, which is perpendicular to the straight, longitudinal axis of the orthopedic implant device 100, 200, 300. (See FIG. 5).

The static bending strength was assessed for multiple embodiments of the orthopedic implant device 100, 200, 300 with a variety of features. Various models of embodiments of the orthopedic implant device 100, 200, 300 configured as an intramedullary nail, as well as models of control intramedullary nails, were generated to assess the static bending strength. The models of controls included non-fenestrated and non-fluted intramedullary nails and fenestrated intramedullary nails. In the models of embodiments of the orthopedic implant device 100, 200, 300 below, the longitudinal flutes were modeled to approximate the longitudinal flutes of existing intramedullary nailing systems, with a total of 4 flutes per nail, each longitudinal flute rotationally offset by 90 degrees.

Each model was loaded to 750 N bending force using the above-described FEA model. As mentioned above, the 750 N bending force was chosen to ensure the model could withstand the forces expected after permanent implantation and weight bearing. The crosshead displacement at this fixed force value, which represents the degree of bending of the nail, was used in this analysis as a proxy for the overall static bending strength of the model. The maximum local stress (the node in the model with the highest stress value) was used as a proxy for the bending fatigue performance of the nail since repeated loading with areas of high local stress may cause an intramedullary nail to fail over time. The naming convention used for these models was “aaOD_bID_cFN_dFL” where aa represents the outer diameter of the intramedullary nail in millimeters, b represents the inner diameter of the nail in millimeters, c represents the diameter of the fenestrations in millimeters, and d represents a binary condition of either having (1) or not having (0) longitudinal flutes.

The analysis was conducted with all models comprising an outer diameter of 9 mm and an inner diameter of 5 mm, with fenestrations of 0 mm (no fenestrations), 1 mm, or 2 mm, each model including or not including standard flutes and the results provided in Table 2. For the purposes of describing Table 2, standard flutes are defined as having a flute width of about 3 mm and the flute radius of the cut is 5 mm. As will be understood, flutes described or claimed herein may have these or other dimensions.

TABLE 2

| | | Max Local | Crosshead |
|---|---|---|---|
| | | Stress | Displacement |
| Model | Dimensions | (MPa) | (mm) |
| 09OD_5ID_0FN_0FL | Outer diameter = 9 mm | 416.6950378 | 0.857095778 |
| | Inner diameter = 5 mm | | |
| | Diameter of | | |
| | fenestrations = 0 mm | | |
| | Includes flutes = no | | |
| 09OD_5ID_0FN_1FL | Outer diameter = 9 mm | 466.8484497 | 1.004648447 |
| | Inner diameter = 5 mm | | |
| | Diameter of | | |
| | fenestrations = 0 mm | | |
| | Includes flutes = yes | | |
| 09OD_5ID_1FN_0FL | Outer diameter = 9 mm | 841.3508301 | 0.879430413 |
| | Inner diameter = 5 mm | | |
| | Diameter of | | |
| | fenestrations = 1 mm | | |
| | Includes flutes = no | | |
| 09OD_5ID_1FN_1FL | Outer diameter = 9 mm | 755.4922485 | 1.016087532 |
| | Inner diameter = 5 mm | | |
| | Diameter of | | |
| | fenestrations = 1 mm | | |
| | Includes flutes = yes | | |
| 09OD_5ID_2FN_0FL | Outer diameter = 9 mm | 879.7621460 | 0.918694258 |
| | Inner diameter = 5 mm | | |
| | Diameter of | | |
| | fenestrations = 2 mm | | |
| | Includes flutes = no | | |
| 09OD_5ID_2FN_1FL | Outer diameter = 9 mm | 780.6137695 | 1.047772408 |
| | Inner diameter = 5 mm | | |
| | Diameter of | | |
| | fenestrations = 2 mm | | |
| | Includes flutes = yes | | |

Referring to Table 2, in some embodiments, the addition of fenestrations has a minor effect on construct deformation/static bending strength. In one or more embodiments, the addition of longitudinal flutes had a large effect on construct deformation/static bending strength. In some embodiments, the presence of longitudinal flutes increased the stress concentration for non-fenestrated nails but reduced the stress concentration for fenestrated nails. In various embodiments, the presence of small fenestrations concentrated the stress less than large fenestrations.

Additionally, various embodiments of the orthopedic implant device 100, 200, 300 including devices with a varying range of outer diameter and inner diameter were tested using the FEA method to determine whether a minor increase in nail outer diameter could offset the effect of fenestrations on fatigue strength relative to commercially available intramedullary nails. The analysis of was conducted with all models comprising varying outer diameter and inner diameter (+0.25 mm or 0.5 mm), with fenestrations of 0 mm (no fenestrations), 1 mm, or 2 mm, each model including or not including standard flutes and the results shown in Table 3. For the purposes of describing Table 3, standard flutes are defined as having a flute width of about 3 mm and the flute radius of the cut is 5 mm. As will be understood, flutes described or claimed herein may have these or other dimensions.

TABLE 3

| | | Max Local | Crosshead |
|---|---|---|---|
| | | Stress | Displacement |
| Model | Dimensions | (MPa) | (mm) |
| 09OD_5ID_0FN_0FL | Outer diameter = 9 mm | 416.6950378 | 0.857095778 |
| | Inner diameter = 5 mm | | |
| | Diameter of | | |
| | fenestrations = 0 mm | | |
| | Includes flutes = no | | |
| 09OD_5ID_0FN_1FL | Outer diameter = 9 mm | 466.8484497 | 1.004648447 |
| | Inner diameter = 5 mm | | |
| | Diameter of | | |
| | fenestrations = 0 mm | | |
| | Includes flutes = no | | |
| 09OD_5ID_1FN_0FL | Outer diameter = 9 mm | 841.3508301 | 0.879430413 |
| | Inner diameter = 5 mm | | |
| | Diameter of | | |
| | fenestrations = 0 mm | | |
| | Includes flutes = no | | |
| 09OD_5ID_1FN_1FL | Outer diameter = 9 mm | 755.4922485 | 1.016087532 |
| | Inner diameter = 5 mm | | |
| | Diameter of | | |
| | fenestrations = 0 mm | | |
| | Includes flutes = no | | |
| 09OD_5ID_2FN_0FL | Outer diameter = 9 mm | 879.762146 | 0.918694258 |
| | Inner diameter = 5 mm | | |
| | Diameter of | | |
| | fenestrations = 0 mm | | |
| | Includes flutes = no | | |
| 09OD_5ID_2FN_1FL | Outer diameter = 9 mm | 780.6137695 | 1.047772408 |
| | Inner diameter = 5 mm | | |
| | Diameter of | | |
| | fenestrations = 0 mm | | |
| | Includes flutes = no | | |
| 09.25OD_5ID_0FN_0FL | Outer diameter = 9 mm | 385.4051514 | 0.771600544 |
| | Inner diameter = 5 mm | | |
| | Diameter of | | |
| | fenestrations = 0 mm | | |
| | Includes flutes = no | | |
| 09.25OD_5ID_0FN_1FL | Outer diameter = 9 mm | 424.5371704 | 0.879794538 |
| | Inner diameter = 5 mm | | |
| | Diameter of | | |
| | fenestrations = 0 mm | | |
| | Includes flutes = no | | |
| 09.25OD_5ID_1FN_0FL | Outer diameter = 9 mm | 712.9852905 | 0.78114897 |
| | Inner diameter = 5 mm | | |
| | Diameter of | | |
| | fenestrations = 0 mm | | |
| | Includes flutes = no | | |
| 09.25OD_5ID_1FN_1FL | Outer diameter = 9 mm | 649.2633667 | 0.889770389 |
| | Inner diameter = 5 mm | | |
| | Diameter of | | |
| | fenestrations = 0 mm | | |
| | Includes flutes = no | | |
| 09.25OD_5ID_2FN_0FL | Outer diameter = 9 mm | 804.4001465 | 0.811008275 |
| | Inner diameter = 5 mm | | |
| | Diameter of | | |
| | fenestrations = 0 mm | | |
| | Includes flutes = no | | |
| 09.25OD_5ID_2FN_1FL | Outer diameter = 9 mm | 771.1242065 | 0.918145299 |
| | Inner diameter = 5 mm | | |
| | Diameter of | | |
| | fenestrations = 0 mm | | |
| | Includes flutes = no | | |
| 09.50OD_5ID_0FN_0FL | Outer diameter = 9.5 mm | 362.9672546 | 0.687404096 |
| | Inner diameter = 5 mm | | |
| | Diameter of | | |
| | fenestrations = 0 mm | | |
| | Includes flutes = no | | |
| 09.50OD_5ID_0FN_1FL | Outer diameter = 9.5 mm | 384.1013794 | 0.776911438 |
| | Inner diameter = 5 mm | | |
| | Diameter of | | |
| | fenestrations = 0 mm | | |
| | Includes flutes = yes | | |
| 09.50OD_5ID_1FN_0FL | Outer diameter = 9.5 mm | 639.9118042 | 0.690383852 |
| | Inner diameter = 5 mm | | |
| | Diameter of | | |
| | fenestrations = 1 mm | | |
| | Includes flutes = no | | |
| 09.50OD_5ID_1FN_1FL | Outer diameter = 9.5 mm | 643.2711182 | 0.785385072 |
| | Inner diameter = 5 mm | | |
| | Diameter of | | |
| | fenestrations = 1 mm | | |
| | Includes flutes = yes | | |
| 09.50OD_5ID_2FN_0FL | Outer diameter = 9.5 mm | 717.4162598 | 0.720021546 |
| | Inner diameter = 5 mm | | |
| | Diameter of | | |
| | fenestrations = 2 mm | | |
| | Includes flutes = no | | |
| 09.50OD_5ID_2FN_1FL | Outer diameter = 9.5 mm | 644.4577637 | 0.810557306 |
| | Inner diameter = 5 mm | | |
| | Diameter of | | |
| | fenestrations = 2 mm | | |
| | Includes flutes = yes | | |
| 10OD_5ID_0FN_0FL | Outer diameter = 10 mm | 297.6520691 | 0.554534376 |
| | Inner diameter = 5 mm | | |
| | Diameter of | | |
| | fenestrations = 0 mm | | |
| | Includes flutes = no | | |
| 10OD_5ID_0FN_1FL | Outer diameter = 10 mm | 319.3292542 | 0.614923954 |
| | Inner diameter = 5 mm | | |
| | Diameter of | | |
| | fenestrations = 0 mm | | |
| | Includes flutes = yes | | |
| 10OD_5ID_1FN_0FL | Outer diameter = 10 mm | 526.2810059 | 0.555695534 |
| | Inner diameter = 5 mm | | |
| | Diameter of | | |
| | fenestrations = 1 mm | | |
| | Includes flutes = no | | |
| 10OD_5ID_1FN_1FL | Outer diameter = 10 mm | 531.7640991 | 0.622128069 |
| | Inner diameter = 5 mm | | |
| | Diameter of | | |
| | fenestrations = 1 mm | | |
| | Includes flutes = yes | | |
| 10OD_5ID_2FN_0FL | Outer diameter = 10 mm | 610.3026733 | 0.575847983 |
| | Inner diameter = 5 mm | | |
| | Diameter of | | |
| | fenestrations = 2 mm | | |
| | Includes flutes = no | | |
| 10OD_5ID_2FN_1FL | Outer diameter = 10 mm | 558.0341187 | 0.641477644 |
| | Inner diameter = 5 mm | | |
| | Diameter of | | |
| | fenestrations = 2 mm | | |
| | Includes flutes = yes | | |

The results of Table 3 demonstrate that the conclusions of the results of Table 2 are consistent across a range of nail outer diameters. Additionally, the results demonstrate that minor increases in nail outer diameter (+0.25 mm or 0.5 mm) may not fully offset the effect of fenestrations on fatigue strength relative to commercially available intramedullary nails. For example, the model 09OD_5ID_0FN_0FL, representative of a 9 mm intramedullary nail with no fenestrations or flutes, has a maximum local stress of 416.7 MPa, while model 09.50OD_5ID_1FN_1FL, representative of a 9.5 mm intramedullary nail with fenestrations with a 1 mm diameter and standard flutes, has a maximum local stress of 643.3 MPa, suggesting there may be an inferior bending fatigue performance in the 9.5 mm intramedullary nail with fenestrations with a 1 mm diameter and standard flutes.

Additionally, multiple embodiments of the orthopedic implant device 100, 200, 300 including devices with a varying range of flute dimensions were tested using the FEA method to determine whether the dimensions of the flutes could be altered to improve the static and fatigue performance of a fenestrated intramedullary nail. The width of the flute and the radius of the cut were varied as shown in in FIG. 4A-D. Nails with each of these flute styles, as well as an unfluted nail, were modeled with 1 mm fenestrations and tested. The results from this analysis are shown in Table 4.

TABLE 4

| | | Max Local | Crosshead |
|---|---|---|---|
| | | Stress | Displacement |
| Model | Dimensions | (MPa) | (mm) |
| 09OD_5ID_1FN_0FL | Outer diameter = 9 mm | 841.3508301 | 0.879430413 |
| | Inner diameter = 5 mm | | |
| | Diameter of | | |
| | fenestrations = 1 mm | | |
| | Includes flutes = no | | |
| 09OD_5ID_1FN_1FL_3W5R | Outer diameter = 9 mm | 755.4922485 | 1.016087532 |
| (FIG. 2A) | Inner diameter = 5 mm | | |
| | Diameter of | | |
| | fenestrations = 1 mm | | |
| | Includes flutes = yes | | |
| 09OD_5ID_1FN_1FL_3W2R | Outer diameter = 9 mm | 779.3065796 | 1.172305465 |
| (FIG. 2B) | Inner diameter = 5 mm | | |
| | Diameter of | | |
| | fenestrations = 1 mm | | |
| | Includes flutes = yes | | |
| 09OD_5ID_1FN_1FL_2W5R | Outer diameter = 9 mm | 668.2755127 | 0.921679735 |
| (FIG. 4C) | Inner diameter = 5 mm | | |
| | Diameter of | | |
| | fenestrations = 1 mm | | |
| | Includes flutes = yes | | |
| 09OD_5ID_1FN_1FL_2W1.1R | Outer diameter = 9 mm | 653.7455444 | 1.012264729 |
| (FIG. 4D) | Inner diameter = 5 mm | | |
| | Diameter of | | |
| | fenestrations = 1 mm | | |
| | Includes flutes = yes | | |

The results of Table 4 demonstrate that reducing the diameter/radius of the arc of the flute cut can substantially improve bending fatigue strength, while preserving static bending strength. Additionally, the results demonstrate that flutes with an increased flute depth further improve maximum local stress, at the cost of decreased static bending strength.

In addition, various embodiments of the orthopedic implant device 100, 200, 300 including devices with a varying range of flute width (FW) of 1 mm, 2 mm, and 3 mm, and varying flute depths (FD) of 0.25 mm, 0.5 mm, and 1 mm, were tested using the FEA method to determine whether additional modifications to the dimensions of the flutes would improve the static and fatigue performance of a fenestrated intramedullary nail. The modeled variations of the orthopedic implant device 100, 200, 300 were assessed relative to a modeled traditional intramedullary nail with flutes (09OD_5ID_0FN_1FL). The results from this analysis are shown in Table 5.

TABLE 5

| | | Max Local | Crosshead |
|---|---|---|---|
| | | Stress | Displacement |
| Model | Dimensions | (MPa) | (mm) |
| 09OD_5ID_0FN_1FL | Outer diameter = 9 mm | 466.8484497 | 1.004648447 |
| | Inner diameter = 5 mm | | |
| | Diameter of | | |
| | fenestrations = 0 mm | | |
| | Includes flutes = yes | | |
| 09OD_5ID_1FN_1FL_1FW_025FD | Outer diameter = 9 mm | 721.465271 | 0.897627532 |
| | Inner diameter = 5 mm | | |
| | Diameter of | | |
| | fenestrations = 1 mm | | |
| | Flute width = 1 mm | | |
| | Flute depth = 0.25 mm | | |
| 09OD_5ID_1FN_1FL_1FW_050FD | Outer diameter = 9 mm | 627.5941772 | 0.919646442 |
| | Inner diameter = 5 mm | | |
| | Diameter of | | |
| | fenestrations = 1 mm | | |
| | Flute width = 1 mm | | |
| | Flute depth = 0.50 mm | | |
| 09OD_5ID_1FN_1FL_1FW_100FD | Outer diameter = 9 mm | 562.1322021 | 0.96523267 |
| | Inner diameter = 5 mm | | |
| | Diameter of | | |
| | fenestrations = 1 mm | | |
| | Flute width = 1 mm | | |
| | Flute depth = 1 mm | | |
| 09OD_5ID_1FN_1FL_2FW_025FD | Outer diameter = 9 mm | 655.5497437 | 0.928542018 |
| | Inner diameter = 5 mm | | |
| | Diameter of | | |
| | fenestrations = 1 mm | | |
| | Flute width = 1 mm | | |
| | Flute depth = 0.25 mm | | |
| 09OD_5ID_1FN_1FL_2FW_050FD | Outer diameter = 9 mm | 651.0325317 | 0.991981447 |
| | Inner diameter = 5 mm | | |
| | Diameter of | | |
| | fenestrations = 1 mm | | |
| | Flute width = 2 mm | | |
| | Flute depth = 0.5 mm | | |
| 09OD_5ID_1FN_1FL_2FW_100FD | Outer diameter = 9 mm | 677.5645142 | 1.066041112 |
| | Inner diameter = 5 mm | | |
| | Diameter of | | |
| | fenestrations = 1 mm | | |
| | Flute width = 2 mm | | |
| | Flute depth = 1 mm | | |
| 09OD_5ID_1FN_1FL_3FW_025FD | Outer diameter = 9 mm | 703.8286743 | 0.914506316 |
| | Inner diameter = 5 mm | | |
| | Diameter of | | |
| | fenestrations = 1 mm | | |
| | Flute width = 3 mm | | |
| | Flute depth = 0.25 mm | | |
| 09OD_5ID_1FN_1FL_3FW_050FD | Outer diameter = 9 mm | 725.6624146 | 1.019451976 |
| | Inner diameter = 5 mm | | |
| | Diameter of | | |
| | fenestrations = 1 mm | | |
| | Flute width = 3 mm | | |
| | Flute depth = 0.5 mm | | |
| 09OD_5ID_1FN_1FL_3FW_100FD | Outer diameter = 9 mm | 733.7736206 | |
| | Inner diameter = 5 mm | | |
| | Diameter of | | |
| | fenestrations = 1 mm | | 1.193047047 |
| | Flute width = 3 mm | | |
| | Flute depth = 1 mm | | |

The results of Table 5 demonstrate that optimal performance, defined as the lowest maximum local stress, while maintaining greater construct static bending strength than the comparable commercially available intramedullary nail, came from the model 09OD_5ID_1FN_1FL_1FW_100FD with the dimensions including an outer diameter of 9 mm, an inner diameter of 5 mm, a diameter of fenestrations of 1 mm, a flute width of 1 mm, and a flute depth of 1 mm. The results of Table 5 show that maximally thin flutes (limited by the outer diameter of the fenestrations) with a depth limited only by the minimum acceptable construct static bending strength provides for the greatest possible bending fatigue performance.

Finally, various embodiments of the orthopedic implant device 100, 200, 300 including devices as described in Table 5 with an outer diameter of 10 mm was tested using the FEA method to determine whether the properties of the embodiments tested in Table 5 would be consistent in an orthopedic implant device with a 10 mm diameter. The results from this analysis are shown in Table 6.

TABLE 6

| | | Max Local | Crosshead |
|---|---|---|---|
| | | Stress | Displacement |
| Model | Dimensions | (MPa) | (mm) |
| 10OD_5ID_0FN_1FL | Outer diameter = 10 mm | 319.3292542 | 0.614923954 |
| | Inner diameter = 5 mm | | |
| | Diameter of | | |
| | fenestrations = 0 mm | | |
| | Includes flutes = yes | | |
| 10OD_5ID_1FN_1FL_1FW_025FD | Outer diameter = 10 mm | 513.0612183 | 0.572999775 |
| | Inner diameter = 5 mm | | |
| | Diameter of | | |
| | fenestrations = 1 mm | | |
| | Flute width = 1 mm | | |
| | Flute depth = 0.25 mm | | |
| 10OD_5ID_1FN_1FL_1FW_050FD | Outer diameter = 10 mm | 491.0563049 | 0.576547742 |
| | Inner diameter = 5 mm | | |
| | Diameter of | | |
| | fenestrations = 1 mm | | |
| | Flute width = 1 mm | | |
| | Flute depth = 0.5 mm | | |
| 10OD_5ID_1FN_1FL_1FW_100FD | Outer diameter = 10 mm | 391.7183228 | 0.605555892 |
| | Inner diameter = 5 mm | | |
| | Diameter of | | |
| | fenestrations = 1 mm | | |
| | Flute width = 1 mm | | |
| | Flute depth = 1 mm | | |
| 10OD_5ID_1FN_1FL_2FW_025FD | Outer diameter = 10 mm | 470.6012573 | 0.58288765 |
| | Inner diameter = 5 mm | | |
| | Diameter of | | |
| | fenestrations = 1 mm | | |
| | Flute width = 2 mm | | |
| | Flute depth = 0.25 mm | | |
| 10OD_5ID_1FN_1FL_2FW_050FD | Outer diameter = 10 mm | 471.1972351 | 0.618884683 |
| | Inner diameter = 5 mm | | |
| | Diameter of | | |
| | fenestrations = 1 mm | | |
| | Flute width = 2 mm | | |
| | Flute depth = 0.5 mm | | |
| 10OD_5ID_1FN_1FL_2FW_100FD | Outer diameter = 10 mm | 454.1640625 | 0.649600744 |
| | Inner diameter = 5 mm | | |
| | Diameter of | | |
| | fenestrations = 1 mm | | |
| | Flute width = 2 mm | | |
| | Flute depth = 1 mm | | |
| 10OD_5ID_1FN_1FL_3FW_025FD | Outer diameter = 10 mm | 538.4782715 | 0.573889017 |
| | Inner diameter = 5 mm | | |
| | Diameter of | | |
| | fenestrations = 1 mm | | |
| | Flute width = 3 mm | | |
| | Flute depth = 0.25 mm | | |
| 10OD_5ID_1FN_1FL_3FW_050FD | Outer diameter = 10 mm | 523.1234741 | 0.628108442 |
| | Inner diameter = 5 mm | | |
| | Diameter of | | |
| | fenestrations = 1 mm | | |
| | Flute width = 3 mm | | |
| | Flute depth = 0.5 mm | | |
| 10OD_5ID_1FN_1FL_3FW_100FD | Outer diameter = 10 mm | 479.3150635 | 0.707045138 |
| | Inner diameter = 5 mm | | |
| | Diameter of | | |
| | fenestrations = 1 mm | | |
| | Flute width = 3 mm | | |
| | Flute depth = 1 mm | | |

The data shown in Table 6 demonstrate that the trends of Table 5 are consistent between fenestrated and fluted orthopedic implant devices with an outer diameter of 9 mm and 10 mm.

In multiple embodiments, the orthopedic implant 100, 200, 300, upon being loaded to 750 N bending force using the above-described FEA model and analyzed according to the methods described in relation to FIG. 5, has a maximum local stress of at least 350 MPa to no more than 600 MPa. In additional instances, the orthopedic implant 100, 200, 300, analyzed according to the methods described in relation to FIG. 5, has a maximum local stress of at least 370 MPa to no more than 565 MPa.

In various instances, the orthopedic implant 100, 200, 300, upon being loaded to 750 N bending force using the above-described FEA model and analyzed according to the methods described in relation to FIG. 5, has a maximum local stress of about 390 MPa. In additional embodiments, the orthopedic implant 100, 200, 300, analyzed according to the methods described in relation to FIG. 5, has a maximum local stress of about 560 MPa. In various instances, the orthopedic implant 100, 200, 300, upon being loaded to 750 N bending force using the above-described FEA model and analyzed according to the methods described in relation to FIG. 5, has a maximum local stress of 391 MPa. In additional embodiments, the orthopedic implant 100, 200, 300, analyzed according to the methods described in relation to FIG. 5, has a maximum local stress of 562 MPa.

Furthermore, in multiple embodiments, the orthopedic implant 100, 200, 300, upon being loaded to 750 N bending force using the above-described FEA model and analyzed according to the methods described in relation to FIG. 5, has a crosshead displacement of at least 0.5 mm to no more than 1 mm. In additional instances, the orthopedic implant 100, 200, 300, analyzed according to the methods described in relation to FIG. 5, has a crosshead displacement of at least 0.5 mm to no more than 1 mm.

In various instances, the orthopedic implant 100, 200, 300, upon being loaded to 750 N bending force using the above-described FEA model and analyzed according to the methods described in relation to FIG. 5, has crosshead displacement about 0.96 mm. In additional embodiments, the orthopedic implant 100, 200, 300, analyzed according to the methods described in relation to FIG. 5, has a crosshead displacement of about 0.6 mm. In various instances, the orthopedic implant 100, 200, 300, upon being loaded to 750 N bending force using the above-described FEA model and analyzed according to the methods described in relation to FIG. 5, has a crosshead displacement of 0.96 mm. In additional embodiments, the orthopedic implant 100, 200, 300, analyzed according to the methods described in relation to FIG. 5, has crosshead displacement of 0.6 mm.

Referring to FIGS. 6A and 6B the addition of certain fenestrations to the configuration of orthopedic implant devices 100, 200, 300 may negatively impact dynamic fatigue performance. In some instances, placement of fenestrations within very thin and deep flutes may minimize the possible decrease in impact dynamic fatigue performance. FIG. 6A illustrates a stress map of embodiments of the orthopedic implant device 100, 200, 300 with thin deep flutes and fenestrations with a diameter of 1 mm. FIG. 6B illustrates a stress map of a representative sample of the orthopedic implant devices 100, 200, 300 with wide and shallow flutes and fenestrations with a diameter of 1 mm. As demonstrated by the hatching in FIGS. 6A and 6B, areas shown as indicating high stress are reduced in FIG. 6A compared to 6B demonstrating that incorporation of fenestrations within very thin and deep flutes in the orthopedic implant devices 100, 200, 300 may decrease the stress and preserve the impact fatigue performance.

An additional benefit of providing various embodiments of the orthopedic implant 100, 200, 300 with thin, deep flutes is that those embodiments may provide ideal shielding from shear forces. As a result, they may improve the feasibility of filling the flutes with temporary carrier loaded with a therapeutic. In many instances, this added feature allows for an initial burst release of the therapeutic from the surface, followed by a consistent, long-term release from the central reservoir through the fenestrations.

### Exemplary Elution Data

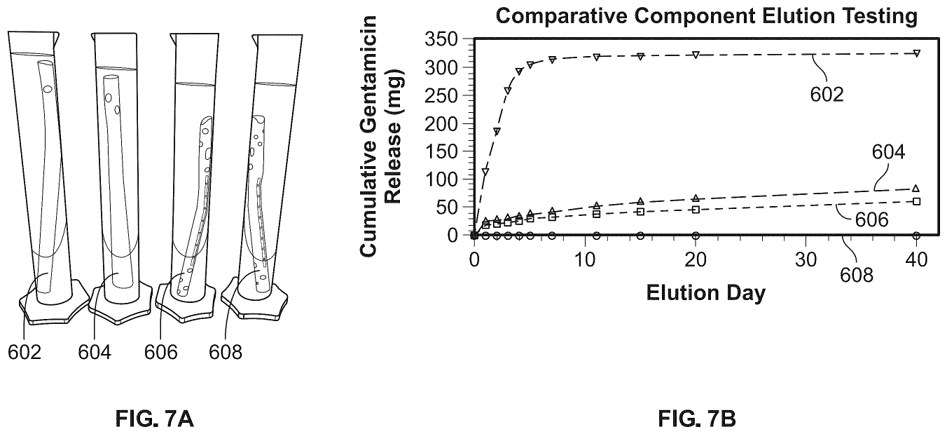

FIGS. 7A and 7B illustrate various intramedullary devices that were analyzed for elution release. FIG. 7A illustrates the intramedullary devices soaked in test cylinders filled with 0.9% normal saline solution and maintained at 37° C. Saline samples were collected from the test cylinders and the concentration of antibiotic was measured (while the volume of soaking saline solution was replaced), in order to determine elution rate.

The intramedullary devices that were tested include: a commercially available intramedullary nail device with an outer diameter of 10 mm and a length of 270 mm coated with a bone cement mixture comprising bone cement powder and gentamicin 602, a commercially available intramedullary nail device with an outer diameter of 10 mm and a length of 260 mm coated with a bone cement mixture comprising bone cement powder, gentamicin and vancomycin 604, a fenestrated and fluted intramedullary nail device with an outer diameter of 10 mm, a central reservoir, the central reservoir having a diameter of 6 mm, a plurality of fenestrations with a diameter of 2 mm, the central reservoir filled with a mixture of calcium sulfate, deionized water, and gentamicin 606, and a fenestrated and fluted intramedullary nail device with an outer diameter of 10 mm, a central reservoir, the central reservoir having a diameter of 6 mm, a plurality of fenestrations with a diameter of 2 mm, the central reservoir filled with a mixture of calcium sulfate and deionized water 608.

FIG. 7B illustrates the comparative component elution testing wherein the commercially available intramedullary nail devices coated with gentamicin or gentamicin and vancomycin (602 and 604 respectively) elute the gentamicin and/or vancomycin at an amount of about 50 mg of antibiotic over at least 40 days. In contrast, the intramedullary nail device with flutes, the plurality of fenestrations, and the central reservoir filled with calcium sulfate and gentamicin 606 has a superior elution profile compared to the commercially available intramedullary nail devices coated with gentamicin or gentamicin and vancomycin (602 and 604 respectively). The intramedullary nail device with flutes, the plurality of fenestrations, and the central reservoir filled with calcium sulfate and gentamicin 606 elutes the gentamicin at an amount of about 300 mg over at least 40 days. The intramedullary nail device with flutes, the plurality of fenestrations, and the central reservoir filled with calcium sulfate 608 serves as a control and shows no elution of gentamicin over time.

### CONCLUSION

The foregoing description of the exemplary embodiments has been presented only for the purposes of illustration and description is not intended to be exhaustive or to limit the devices, systems, methods, and apparatuses herein to the precise forms disclosed. Many modifications and variations are possible considering the above teachings.

The embodiments were chosen and described in order to explain the principles of the technology discussed herein and their practical application to enable others skilled in the art to utilize the various embodiments and with various modifications as are suited to the particular use contemplated. Alternative embodiments will become apparent to those skilled in the art to which the present technologies pertain without departing from their spirit and scope.

What is claimed is:

1. An orthopedic implant device comprising:
a longitudinal axis;
a diameter of about 7 mm to about 15 mm;
a proximal end;
a distal end;
a device wall;
a straight region comprising:
one or more concave longitudinal flutes oriented parallel to the longitudinal axis, each longitudinal flute comprising:
a bottom surface;
a flute length of about 100 mm to about 450 mm;
a flute width of about 1 mm to about 3 mm;
a flute depth of about 0.25 mm to about 1 mm; and
a plurality of fenestrations through the bottom surface and extending into a central reservoir, each fenestration comprising a diameter of about 0.5 mm to about 3 mm; and
at least one screw hole with a diameter of about 5 mm located near the distal end,
wherein the device wall has a thickness of about 2 mm to about 4 mm and the central reservoir is configured for holding a volume of about 4 mL to about 9 mL of a temporary carrier loaded with at least one therapeutic.

2. The orthopedic implant device of claim 1, wherein the orthopedic implant device is an intramedullary nail.

3. The orthopedic implant device of claim 2, wherein the central reservoir is filled prior to intraoperative use with the temporary carrier loaded with at least one therapeutic.

4. The orthopedic implant device of claim 2, wherein the central reservoir is filled during intraoperative use with the temporary carrier loaded with at least one therapeutic.

5. The orthopedic implant of claim 2, wherein the fenestrations are evenly spaced within each flute along the longitudinal axis.

6. The orthopedic implant device of claim 5, wherein the fenestrations are orthogonally oriented and offset in spacing.

7. The orthopedic implant device of claim 6, wherein the central reservoir has a diameter of about 5 mm.

8. The orthopedic implant device of claim 6, wherein at a global force level of 750 N, the implant has a maximum local stress of about 350 to about 600 MPa.

9. The orthopedic implant device of claim 1, wherein the temporary carrier is calcium sulfate loaded with gentamicin and is configured for elution over about 40 days.

10. The orthopedic implant device of claim 1, wherein the temporary carrier is calcium sulfate loaded with vancomycin and is configured for elution over about 40 days.

11. The orthopedic implant device of claim 1, wherein the temporary carrier is calcium sulfate loaded with tobramycin and is configured for elution over about 40 days.

12. The orthopedic implant device of claim 1, wherein the temporary carrier is calcium sulfate loaded with gentamicin and vancomycin and is configured for elution over about 40 days.

13. The orthopedic implant device of claim 1, wherein the temporary carrier is calcium sulfate loaded with tobramycin and vancomycin and is configured for elution over about 40 days.

14. An orthopedic implant device comprising:
a longitudinal axis;
a diameter of about 7 mm to about 15 mm;
one or more radially distributed concave longitudinal flutes oriented parallel to the longitudinal axis, each longitudinal flute comprising:
a bottom surface;
a width of about 1 mm to about 3 mm;
a depth of about 0.25 mm to about 1 mm; and
a plurality of fenestrations through the bottom surface and extending into a central reservoir, each fenestration comprising a diameter of about 0.5 mm to about 3 mm; and
at least one screw hole, wherein the central reservoir is configured for holding a volume of about 4 mL to about 9 mL of a temporary carrier loaded with at least one therapeutic.

15. The orthopedic implant device of claim 14, wherein the device is an intramedullary nail.

16. The orthopedic implant device of claim 14, wherein the central reservoir is filled prior to intraoperative use with the temporary carrier loaded with at least one therapeutic.

17. The orthopedic implant device of claim 14, wherein the central reservoir is filled during intraoperative use with the temporary carrier loaded with at least one therapeutic.

18. The orthopedic implant device of claim 15, wherein each longitudinal flute is about 1 mm wide and about 1 mm deep.

19. The orthopedic implant device of claim 15, wherein the plurality of fenestrations have a diameter of about 1 mm.

20. The orthopedic implant device of claim 15, wherein the plurality of fenestrations are evenly spaced within each flute along the longitudinal axis.

21. The orthopedic implant device of claim 20, wherein the plurality of fenestrations are orthogonally oriented and offset in spacing.

22. The orthopedic implant device of claim 20, wherein the central reservoir has a diameter of about 5 mm.

23. The orthopedic implant device of claim 14, wherein at a global force level of 750 N, the implant has a maximum local stress of about 350 to about 600 MPa.

24. The orthopedic implant device of claim 14, wherein the temporary carrier is calcium sulfate loaded with at least gentamicin, vancomycin, or tobramycin and is eluted over about 40 days.

25. The orthopedic implant device of claim 14, wherein the temporary carrier is calcium sulfate loaded with gentamicin and vancomycin and is eluted over about 40 days.

26. The orthopedic implant device of claim 14, wherein the temporary carrier is calcium sulfate loaded with vancomycin and tobramycin and is eluted over about 40 days.

27. An orthopedic implant device comprising:
a longitudinal axis;
a diameter of about 7 mm to about 15 mm;
one or more concave flutes oriented radially down the length to the longitudinal axis, each flute comprising:
a bottom surface;
a width of about 1 mm to about 3 mm;
a depth of about 0.25 mm to about 1 mm; and
a plurality of fenestrations through the bottom surface and extending into a central reservoir, each fenestration comprising a diameter of about 0.5 mm to about 3 mm; and
at least one screw hole, wherein the central reservoir is configured for holding a volume of about 4 mL to about 9 mL of a temporary carrier loaded with at least one therapeutic.

28. The orthopedic implant device of claim 27, wherein the device is an intramedullary nail.

29. An orthopedic implant device comprising:
a straight region comprising:
an external surface having a plurality of fenestrations,
one or more concave longitudinal flutes oriented parallel to the longitudinal axis and comprising:
a bottom surface; and
a plurality of fenestrations through the bottom surface; and
a central reservoir filled with a temporary carrier and connected to the plurality of fenestrations, wherein the orthopedic implant device is configured to elute the temporary carrier over about 40 days.

30. The orthopedic implant device of claim 29, wherein the concave longitudinal flutes are radially distributed around the longitudinal axis.
